(19) United States
(12) Patent Application Publication
(10) Pub. No.: US 2026/0014062 A1
(43) Pub. Date: Jan. 15, 2026

(54) COMPOSITIONS SUITABLE AS SETTERS

(71) Applicant: L'OREAL, PARIS (FR)

(72) Inventors: YIXIN YANG, UNION, NJ (US); ANNE-LAURE SUZANNE BERNARD, NEW YORK, NY (US); BRADY ZARKET, EDISON, NJ (US)

(73) Assignee: L'OREAL, Paris (FR)

(21) Appl. No.: 19/338,274

(22) Filed: Sep. 24, 2025

Related U.S. Application Data
(62) Division of application No. 17/731,712, filed on Apr. 28, 2022.

(51) Int. Cl. A61K 8/34; A61K 8/04; A61K 8/49; A61K 8/67; A61K 8/85; A61Q 1/00

(52) U.S. Cl. CPC A61K 8/34; A61K 8/042; A61K 8/4926; A61K 8/676; A61K 8/85; A61Q 1/00; A61K 2800/30; A61K 2800/884

(57) ABSTRACT

The invention relates to compositions, in particular setter compositions for cosmetics, including (1) at least one polyphenol X comprising at least two different phenol groups; (2) at least one (poly)glycerolated and/or polyoxyalkylenated nonionic compound Y; (3) a solvent system including (i) water in an amount of at least about 10% by weight with respect to the total weight of the composition; and (ii) at least one C2-C5 monoalcohol in an amount effective to inhibit formation of a precipitate of compound X and compound Y in the composition prior to application; (4) at least one chelating agent; and (5) at least one ascorbic acid compound, as well as to methods, kits and sets comprising such compositions in container(s) or in application.

### FIELD OF THE INVENTION

The present invention relates to compositions, in particular setter compositions for cosmetics, comprising (1) at least one polyphenol X comprising at least two different phenol groups; (2) at least one (poly)glycerolated and/or polyoxyalkylenated nonionic compound Y; (3) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application; (4) at least one chelating agent; and (5) at least one ascorbic acid compound, as well as to methods, kits and sets comprising such compositions in container(s) or in application.

### DISCUSSION OF THE BACKGROUND

At the present time on the market for caring for and making up keratin materials, many products claim staying power throughout the day, withstanding external factors such as water, sebum, mechanical friction, etc. (waterproof mascara, food-proof lipsticks, long-lasting foundations). Long-lasting products for the lips, the eyelashes, the eyebrows or the face, which can be used at home, are mainly based on synthetic coating polymers in the presence of organic solvents. For making up the lips and compositions for making up the face, compositions comprising a silicone resin as coating agent are known, such as the compound having the INCI name: Trimethylsiloxysilicate or a compound having the INCI name: Polypropylsilsesquioxane, or alternatively a silicone acrylate copolymer such as the product having the INCI name:

Acrylates/polytrimethylsiloxymethacrylate copolymer). Long-lasting makeup products for the eyelashes and/or the eyebrows (mascaras, eyeliners) use, for their part, waxes or film-forming polymer particles in aqueous suspension of the latex type (i.e.: styrene/acrylate copolymers).

Besides these long-lasting products, the current trend is towards semi-permanent makeup. Specifically, in recent years, conventional makeup products have met with competition from the market of semi-permanent makeup in professional salons. It is encountered in the sector of makeup for the eyes (semi-permanent mascara, permanent eyelash makeup, eyelash extensions, etc.), for the eyebrows (semi-pigmentation known as micro-blading), for the complexion (freckles, beauty spots or the whole face, glowing or healthy-complexion effect) or for the lips (semi-permanent tattooing). This new trend is driving consumers towards seeking increasingly long staying power for greater practicality (avoiding having to apply and remove makeup daily, healthy complexion effect immediately on waking up, etc.).

However, consumers, who are increasingly demanding as regards the composition of their cosmetic products, are also seeking to use products with ingredients that are well tolerated such as natural ingredients, with ingredients which have little or no environmental impact and/or ingredients which are compatible with numerous packagings.

The aim of the present invention is to propose compositions which offer excellent staying power of the expected cosmetic effects, notably the color of the makeup on keratin materials (skin, lips, nails, hair, eyelashes, eyebrows) which may extend the duration of the cosmetic composition on keratin materials, as well as improve wear of the cosmetic composition on keratin materials and its resistance to external forces such as mechanical friction, water, sweat and perspiration, sebum, oil, etc.

In addition, the aim of the present invention is to propose compositions which afford staying power of the expected cosmetic effects, notably the colour of the makeup on keratin materials, combined with a good level of comfort in comparison with conventional systems, in particular based on silicone resin.

Use of tannic acid in compositions has been reported. For example, CN104971200 is titled “Tannic acid pH-stable hemorrhoid gel and preparation method thereof,” and describes as its object as providing a tannic acid pH stable hemorrhoid gel such that when the gel contacts weakly alkaline anal tissue and weakly alkaline stool, still can maintain pH=4.5-4.8, so that tannic acid and other raw materials synergize, play a better role in astringent healing of hemorrhoids.

Also, CN108175765 is titled “Acid-sensitive controlled-release anti-inflammatory gel as well as preparation method and application thereof,” where the purpose is to design a hyaluronic acid hydrogel structure according to the characteristics of pH drop caused by the accumulation of inflammatory metabolites in the joint cavity, to provide an acid-sensitive controlled-release anti-inflammatory gel which is formed by phenylboronic acid-modified hyaluronic acid and tannic acid under neutral to weakly alkaline conditions.

U.S. Pat. No. 8,377,853 (corresponding to CA2649412) is titled “Aqueous gels for well bore strengthening” and discloses in the abstract “A process for treating an earth formation is disclosed, the process may include: injecting a gelling agent into the earthen formation; injecting a crosslinking agent into the earthen formation; and reacting the gelling agent and the crosslinking agent to form a gel. The gelling agent may include at least one of a lignin, a lignosulfonate, a tannin, a tannic acid, a modified lignin, a modified lignosulfonate, a modified tannin, a modified tannic acid, biopolymers, starches, carboxy methyl cellulose, polyacrylates, polyacrylamides, polyamines, polyether amines, poly vinyl amines, polyethylene imines, and combinations thereof. The crosslinking agent may include at least one of ethylene glycol diglycidyl ether, propylene glycol diglycidyl ether, butylene glycol diglycidyl ether, sorbitol polyglycidyl ether, trimethylolpropane triglycidyl ether, sorbitol polyglycidyl ether, diglycidyl ether of neopentyl glycol, epoxidized 1,6-hexanediol, aziridine derivatives, epoxy functionalized polyalkalene glycols, an oxidized starch, a polymeric dialdehyde, an aldehyde adduct, a tetra methoxy propane, a hydrolized acetal, and combinations thereof.”

Further, general stabilizing effects of compositions using various ingredients has also been reported, such as in Effect of Sodium Metabisulphite and Disodium Ethylenediaminetetraacetic acid (EDTA) on the Stability of Ascorbic Acid in Vitamin C Syrup, Researcher 2014; 6 (10), which relates to using EDTA and sodium meta bisuiphite and additional ascorbic acid to improve Vitamin C syrup stability.

There remains a need for improved compositions such as setter compositions having improved properties with respect to composition ease of and/or comfort of application of the composition, composition stability, and/or composition color stability, and in particular with respect to improved properties related to the presence of polyphenols such as tannic acid in the compositions including properties such as improved polyphenol stability against oxidation and/or hydrolysis leading to aggregation of byproducts.

Accordingly, one aspect of the present invention is a composition, in particular a setter composition, which has improved properties with respect to composition ease of and/or comfort of application of the composition, composition stability, and/or composition color stability, and in particular with respect to improved properties related to the presence of polyphenols such as tannic acid in the compositions including properties such as improved polyphenol stability against oxidation and/or hydrolysis leading to aggregation of byproducts.

### SUMMARY OF THE INVENTION

The present invention relates to compositions, in particular setter compositions for cosmetics, comprising (1) at least one polyphenol X comprising at least two different phenol groups, (2) at least one (poly)glycerolated and/or polyoxyalkylenated nonionic compound, (3) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application, (4) at least one chelating agent, and (5) at least one ascorbic acid compound. Preferably, the composition further comprises at least one gelling agent. Also preferably, the composition is a gel composition and/or has a pH less than 7.

The present invention also relates to methods of caring for, and/or making up keratinous material by applying compositions of the present invention to the keratinous material in an amount sufficient to care for, and/or to make up the keratinous material.

The present invention also relates to methods of setting a cosmetic composition (e.g., color coat composition) and/or priming keratinous material for a cosmetic composition (e.g., color coat composition) by applying compositions of the present invention to the keratinous material (priming) and/or to a cosmetic composition (e.g., color coat composition) previously-applied to keratinous material (setting) in an amount sufficient to prime the keratinous material for the cosmetic composition and/or in an amount sufficient to obtain setting of the previously-applied cosmetic composition to keratinous material.

The present invention also relates to kits comprising, as separate compositions in one or more containers within the kits, (A) a composition, in particular a setter composition for cosmetics, comprising (1) at least one polyphenol X comprising at least two different phenol groups, (2) at least one (poly)glycerolated and/or polyoxyalkylenated nonionic compound Y, (3) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application, (4) at least one chelating agent, and (5) at least one ascorbic acid compound; and (B) at least one color coat composition comprising at least one colorant. Preferably, the composition further comprises at least one gelling agent. Also preferably, the composition is a gel composition and/or has a pH less than 7.

The present invention also relates to sets, as applied onto keratinous material, comprising (A) at least one first layer of at least one color coat composition; and (B) at least one second layer of at least one composition, in particular a setter composition for cosmetics, comprising (1) at least one polyphenol X comprising at least two different phenol groups, (2) at least one (poly)glycerolated and/or polyoxyalkylenated nonionic compound Y, (3) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application, (4) at least one chelating agent, and (5) at least one ascorbic acid compound. Preferably, the at least one second layer is applied over the at least one first layer (in which case the second layer is a setter layer formed by application of a setter composition to the first layer). However, if the at least one first layer is applied over the at least one second layer, the at least one second layer is a primer layer formed by application of a primer composition over keratinous material). Preferably, the composition further comprises at least one gelling agent. Also preferably, the composition is a gel composition and/or has a pH less than 7.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of triethanolamine (TEA)” means that an effective amount (that is, more than trace amounts) of TEA is omitted from the composition (that is, about 0% by weight), “substantially free of TEA” means that TEA is are present in amounts not greater than 0.1% by weight, and “devoid of TEA” means that TEA is present in amounts not greater than 0.25% by weight, based on the total weight of the composition. The same nomenclature applies for all other ingredients identified throughout the application and in this paragraph such as, for example, oils (compositions of the invention which are “free of oils,” “substantially free of oils,” and “devoid of oils” have meanings consistent with the discussion within this paragraph), even if not specifically discussed for each identified ingredient. Discussed examples of the use of such language are intended to be exemplary, not limiting.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as lips and evaluating the color of the composition after an extended period of time. For example, the color of a composition may be evaluated immediately following application to keratin materials such as lips and these characteristics may then be re-evaluated and compared after a certain amount of time. Further, these characteristics may be evaluated with respect to other compositions, such as commercially available compositions.

“Keratinous material” or “keratin material” means natural nails, lips, skin such as the face, the body, the hands, and the area around the eyes, and keratin fibres such as head hair, eyelashes, eyebrows, bodily hair of a human, as well as synthetic additions such as false eyelashes, false eyebrows, false nails, etc.

“Physiologically acceptable” means compatible with keratinous material and having a pleasant color, odor and feel, and which does not cause any unacceptable discomfort (stinging or tautness) liable to discourage a consumer from using the composition. Acceptable PH levels for compositions of the present invention are preferably acidic, that is, less than 7, preferably 6.5 or less, preferably 6.0 or less, preferably 5.5 or less, including all ranges and subranges therebetween such as, for example 3 to 5, 4 to 6, 3 to 4.5, etc. Compositions of the present invention may also be in the form of a gel composition.

“Gel composition” means a composition which does not flow similar to a liquid when applied to a substrate, a composition which has a 3-dimensional network that inhibits the composition from spreading on, or dripping from, a substrate after application owing to gravity over short period of time (e.g. less than 10 seconds). G′ (storage modulus) is higher than G″ (loss modulus) at all range of strains up to 300% strain. Preferably, G′ (storage modulus) is higher than G″ (loss modulus) at low strain, but with G′ decreasing and G″ increasing, the a gel has the crossover point at >0.1% strain, preferably >1% strain, and preferably less than 200% strain, preferably less than 150% strain.

“Gel Crossover Point” (Sol/Gel Point), means the point at which the G″ (loss modulus) intersects the G′ (storage modulus), reported in % strain. It is the point at which a composition goes from a more solid state to a more liquid state. An example of a method for determining gel crossover point is as follows: G″ (loss modulus) and G′ (storage modulus) using a Discovery HR-3 Rheometer by TA Instruments, having 40 mm parallel plate geometry on a stainless steel flat peltier plate. The test can be run @ 25° C., with test parameter of angular frequency of 1.0 rad/s and logarithmic sweep: Strain % 0.01 to 1000.0%. 5 points per decade. Results reported in % strain.

“Hydrogen bonding interaction” means an interaction involving a hydrogen atom of one of the two reagents and an electronegative heteroatom of the other reagent, such as oxygen, nitrogen, sulfur and fluorine. In the context of the invention, the hydrogen bonding forms between the hydroxyl functions (OH) of the reactive phenol groups of the polyphenol X and the reactive hydroxyl groups of the compound Y, which are capable of forming hydrogen bonding with those of said phenol groups of the polyphenol X.

“Coating agent formed by interaction by hydrogen bonds of at least one polyphenol X comprising at least two different phenol groups with the compound Y” means that the conditions are met so that the reaction can be carried out between the two reagents, in particular that:
i) the amount of polyphenol X is sufficient in the composition containing it, and ii) the compound Y is soluble, miscible or solubilized by another solvent in the medium of the composition containing it, and iii) the compound Y has a sufficient number of hydrogen bond acceptor groups to react with the phenol groups of polyphenol X and, in the medium of the composition containing it, and iv) the compound Y, in the medium of the composition containing it, does not comprise in its structure any group which does not allow the formation of hydrogen bonding with the functions of the reactive phenol groups of the polyphenol X, such as for example, one or more anionic group.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

“Coating agent” refers to any compound which is capable of forming a deposit on the surface of a keratin material to which it has been applied.

“Hydrogen bonding-inhibiting agent” refers to any compound which is capable of preventing hydrogen bonding interaction between the polyphenol X and the compound Y and/or which is capable of dissociating the complex formed by said interaction by breaking the hydrogen bonding.

“Precipitate” or “precipitation” refers to the result of hydrogen bonding interaction between the polyphenol X and the compound Y in the composition.

“Aggregate” or “aggregation” refers to the result of decomposition of polyphenol X in the composition owing to reactions such as hydrolysis and/or oxidation.

The compositions and methods of the present invention can comprise, consist of, or consist essentially of the essential elements and limitations of the invention described herein, as well as any additional or optional ingredients, components, or limitations described herein or otherwise useful. For example, the solvent system can “consist essentially of” water and C2-C5 monoalcohol, and/or the surfactant system can “consist essentially of” identified surfactant(s) or types of surfactants discussed below.

For purposes of the present invention, the “basic and novel property” associated with compositions, components and methods which “consist essentially of” identified ingredients or actions is “polyphenol stability as evidenced by reduced amount of color darkening and aggregation as defined above. Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Polyphenol X

According to the present invention, compositions comprising at least one polyphenol X comprising at least two different phenol groups are provided.

The polyphenols X that may be used according to the present invention include in their structure at least two different phenol groups.

The term “polyphenol” refers to any compound containing in its chemical structure at least two and preferably at least three phenol groups.

The term “phenol group” refers to any group comprising an aromatic ring, preferably a benzene ring, including at least one hydroxyl group (OH).

The term “different phenol groups” refers to phenol groups that are chemically different.

The polyphenols X that may be used according to the invention may be synthetic or natural. They may be in isolated form or contained in a mixture, notably contained in a plant extract. Polyphenols are phenols comprising at least two phenol groups that are differently substituted on the aromatic ring.

The two classes of polyphenols are flavonoids and non-flavonoids.

Examples of flavonoids that may be mentioned include chalcones such as phloretin, phloridzin, aspalathin or neohesperidin; flavanols such as catechin, fisetin, kaempferol, myricetin, quercetin, rutin, procyanidins, proanthocyanidins, pyroanthocyanidins, theaflavins or thearubigins (or thearubrins); dihydroflavonols such as astilbin, dihydroquercetin (taxifolin) or silibinin; flavanones such as hesperidin, neohesperidin, hesperetin, naringenin or naringin; anthocyanins such as cyanidin, delphinidin, malvidin, peonidin or petunidin; catechin tannins such as tannic acid; isoflavonoids such as daidzein or genistein; neoflavanoids; lignans such as pyroresorcinol; and mixtures thereof.

Among the natural polyphenols that may be used according to the invention, mention may also be made of lignins.

Examples of non-flavonoids that may be mentioned include curcuminoids such as curcumin or tetrahydrocurcumin; stilbenoids such as astringin, resveratrol or rhaponticin; aurones such as aureusidin; and mixtures thereof.

As polyphenols that may be used according to the invention, mention may also be made of chlorogenic acid, verbascoside; coumarins substituted with phenols.

According to a particular embodiment of the invention, the polyphenol X will be chosen from catechin tannins such as gallotannins chosen from tannic acid; ellagitannins such as epigallocatechin, epigallocatechin gallate, castalagin, vescalagin, vescalin, castalin, casuarictin, castanopsinins, excoecarianins, grandinin, gradinin, roburins, pterocarinin, acutissimin, tellimagrandins, sanguiin, potentillin, pedunculagin, geraniin, chebulagic acid, repandisinic acid, ascorgeraniin, stachyurin, casuarinin, casuariin, punicacortein, coriariin, cameliatannin, isodeshydrodigalloyl, dehydrodigalloyl, hellinoyl, punicalagin and rhoipteleanins.

According to a particular embodiment of the invention, the polyphenol X is epigallocatechin, in particular a green tea extract having the INCI name Green Tea Extract, notably comprising at least 45% epigallocatechin relative to the total weight of said extract, for instance the commercial product sold under the name Dermofeel Phenon 90 M-C® sold by the company Evonik Nutrition & Care or the commercial product sold under the name Tea Polyphenols Green Tea Extract® by the company Tayo Green Power.

According to a particular embodiment of the invention, the polyphenol X is a procyanidin or a mixture of procyanidins, in particular an extract of maritime pine bark having the INCI name Pinus pinaster Bark/Bud Extract, notably comprising at least 65% by weight of procyanidins relative to the total weight of said extract, such as the commercial product sold under the name Pycnogenol® sold by the company Biolandes Arômes.

Tannic acid will be used more particularly as polyphenol X.

According to a particular embodiment, the polyphenol(s) X according to the invention is preferably present in a content equal or greater than 0.5% by weight, preferably equal or greater than 0.8% by weight, preferably equal or greater than 1% by weight, preferably equal or greater than 2% by weight relative to the total weight of the composition containing it (them).

According to a particular embodiment, the polyphenol(s) X according to the invention is preferably present in a content from about 0.5 to about 30% by weight, preferably from about 0.75 to about 25% by weight, preferably from about 1 to about 25% by weight, and preferably from about 2 to about 15% by weight, relative to the total weight of the composition containing it (them), including all ranges and subranges therebetween such as, for example, 3 to 8% by weight, 0.5% to 7.5% by weight, 2 to 5% by weight, etc.

### Nonionic Compound Y

According to the present invention, compositions comprising at least one (poly)glycerolated and/or polyoxyalkylenated nonionic compound Y are provided. Preferably, nonionic compound Y has a molar mass greater than 200 g/mol.

For the purposes of the present invention, the term “polyoxyalkylenated compound” refers to any molecule comprising in its chemical structure at least chain comprising oxyalkylene units, in particular oxyethylene units —(OCH₂CH₂)ₙ and/or oxypropylene units-(OCH₂CH₂CH₂)ₚ, where n and p are 2 or greater.

For the purposes of the present invention, the term “polyglycerolated compound” refers to any molecule comprising in its chemical structure a glycerol group or a chain comprising glycerol units —(O—CH₂—CHOH—CH₂)ₘ, where m is 2 or greater; “glycerolated compound” refers to any molecule comprising in its chemical structure a glycerol group or a chain comprising glycerol units —(O—CH₂—CHOH—CH₂)ₘ, where m is 1; and “(poly)glycerolated compound” refers to any molecule comprising in its chemical structure a glycerol group or a chain comprising glycerol units —(O—CH₂—CHOH—CH₂)ₘ, where m is 1 or greater.

In one preferred embodiment, the molar mass of the compound Y is greater than 350 g/mol.

As examples of monoglycerolated or (poly)glycerolated or polyoxyalkylenated nonionic compounds Y, mention may be made of:
(1) Glycerolated alkyl ethers such as glyceryl lauryl ether,
(2) (poly)glycerolated alkyl ether nonionic surfactants, in particular chosen from polyglyceryl-2 oleyl ether and polyglyceryl-4 oleyl ether,
(3) Glycerol or polyglycerol esters of fatty acids, which are optionally polyhydroxylated, in particular chosen from polyglyceryl-3 polyricinoleate, polyglyceryl-2 diisostearate, polyglyceryl-4 diisostearate, polyglyceryl-4 caprate, polyglyceryl-2 stearate, polyglyceryl-3 dicitrate/stearate, polyglyceryl-10 dioleate, polyglyceryl-3 diisostearate, polyglyceryl-2 triisostearate, polyglyceryl-10 laurate, glyceryl stearate citrate and polyglyceryl-2 dipolyhydroxystearate,
(4) Polyoxyethylenated or (poly)glycerolated waxes, notably chosen from polyoxyethylenated ester waxes such as polyoxyethylenated jojoba wax, PEG-8 Beeswax, PEG-60 Lanolin, PEG-75 Lanolin, PPG-12-PEG-50 Lanolin and Polyglyceryl-3 Beeswax,
(5) Polyethylene glycols of the type H(O—CH₂—CH₂)ₙ—OH, in particular chosen from PEG-6, PEG-8, PEG-14M, PEG-20, PEG-45M, PEG-90, PEG-90M, PEG-150, PEG-180 and PEG-220,
(6) Poloxamers of the type HO—(CH₂—CH₂—O)ₙ—(CHCH₃—CH₂—O)O—(CH₂—CH₂—O)ₚ—H,
(7) Polypropylene glycol alkyl ethers of the type: CₙH₂ₙ₊₁—(O—C(CH₃)H—CH₂)ₒ—(O—CH₂—CH₂)ₚ—OH in particular chosen from PPG-26-Buteth-26, PPG-5-Ceteth-20 and PPG-6-Decyltetradeceth-30,
(8) Compounds of the type: H(O—C(CₙH₂ₙ₊₁)—CH₂)ₒ—(CH₂—CH₂—O)ₚ—(CH₂—C(C_{q}H_{2q+1})H—O)ᵣH in particular PEG-45/Dodecyl Glycol Copolymer.
(9) Compounds of the type: CₙH₂ₙ₊₁—(O—CH₂—CH₂)ₒ—O—CH₂—C(CₚH₂ₚ₊₁)HOH in particular Ceteareth-60 Myristyl Glycol,
(10) Polyoxyethylenated glycerols, in particular glycerol oxyethylenated with 26 OE (Glycereth-26),
(11) Alkylpolyethylene glycols of the type CₙH₂ₙ₊₁—(O—CH₂—CH₂)ₒ—OH, in particular chosen from Ceteth-2, Ceteth-10, Ceteth-20, Ceteth-25, Isoceteth-20, Laureth-2, Laureth-3, Laureth-4, Laureth-12, Laureth-23, Oleth-2, Oleth-5, Oleth-10, Oleth-20, Oleth-25, Deceth-3, Deceth-5, Beheneth-10, Steareth-2, Steareth-10, Steareth-20, Steareth-21, Steareth-100, Ceteareth-12, Ceteareth-15, Ceteareth-20, Ceteareth-25, Ceteareth-30, Ceteareth-33, Coceth-7 and Trideceth-12,
(12) Polyoxyethylenated alkylamines of the type CH₃—(CH₂)ₙ—(CH═CH)ₒ—(CH)ₚ—N((CH₂—CH₂—O)H)_{q}((CH₂—CH₂—O)ᵣH), in particular: PEG-2-Oleamine,
(13) Fatty acid esters of polyethylene glycol of the type CₙH₂ₙ₊₁—(CH═CH₂)ₒ—CₚH₂ₚ—CO—(O—CH₂—CH₂)ₙ—OH or CₙH₂ₙ₊₁—(CH═CH)ₒ—CₚH₂ₚ—CO—(O—CH₂—CH₂)_{q}—O—CO—CᵣH₂ᵣ₊₁ or CₙH₂ₙ₊₁—(CH═CH)ₒ—CO—(O—CH₂—CH₂)_{q}—O—CₙH₂ₙ₊₁ or CₙH₂ₙ₊₁—O—CH(alkyl)-(CH₂)ₚ—(O—CH₂—CH₂)_{q}—O—CO—CᵣH₂ᵣ₊₁ in particular chosen from PEG-6 Isostearate, PEG-6 Stearate, PEG-8 Stearate, PEG-8 Isostearate, PEG-20 Stearate, PEG-30 Stearate, PEG-32 Stearate, PEG-40 Stearate, PEG-75 Stearate, PEG-100 Stearate, PEG-8 Distearate, PEG-150 Distearate, Mereth-3 Myristate, PEG-4 Olivate, Propylene Glycol Ceteth-3 Acetate and PEG-30 Dipolyhydroxystearate,
(14) Polyoxyethylenated alkylglycerides, in particular chosen from PEG-6 Caprylic/Capric Glycerides, PEG-60 Almond Glycerides, PEG-10 Olive Glycerides and PEG-45 Palm Kernel Glycerides,
(15) Polyoxyethylenated alkylglucoses, in particular chosen from Methyl-Gluceth-10 and Methyl-Gluceth-20,
(16) Polyoxyethylenated sugar esters such as PEG-120 Methyl Glucose Dioleate or PEG-20 Methyl Glucose Sesquistearate,
(17) Polyoxyalkylenated alkyl glycol ethers such as PPG-1-PEG-9 Lauryl Glycol Ether,
(18) Polyoxyethylenated or (poly)glycerolated pentaerythritol esters and ethers, in particular chosen from PEG-150 Pentaerythrityl Tetrastearate,
(19) Polysorbates, in particular chosen from Polysorbate-20, Polysorbate-21, Polysorbate-60, Polysorbate-61, Polysorbate-80 and Polysorbate-85,
(20) Polyoxyethylenated polyamines, in particular PEG-15 Cocopolyamine,
(21) Polyoxyethylenated dihydrocholesteryl esters of structure:

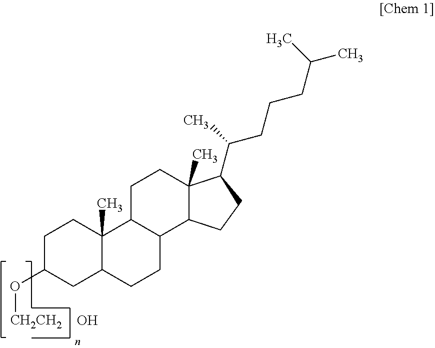

in particular Dihydrocholeth-30,
(22) Polyoxyethylenated ingredients chosen from the mixture of polyoxyethylenated (200 OE) palm glycerides and of polyoxyethylenated (7 OE) coconut kernel oil, PEG-7 Glyceryl Cocoate, PEG-30 Glyceryl Cocoate, PEG-40 Hydrogenated Castor Oil, PEG-60 Hydrogenated Castor Oil, PEG-30 Glyceryl Stearate, PEG-200 Glyceryl Stearate, PEG-20 Glyceryl Triisostearate, PEG-55 Propylene Glycol Oleate, PEG-70 Mango Glycerides, Hydrogenated Palm/Palm Kernel Oil PEG-6 Esters, PEG-200 Hydrogenated Glyceryl Palmitate and PEG-7 Glyceryl Cocoate,
(23) Polyoxyethylenated butters, in particular polyoxyethylenated shea butter,
(24) Polyoxyalkylenated and/or (poly)glycerolated silicones, in particular chosen from PEG/PPG-17/18 Dimethicone, PEG/PPG-18/18 Dimethicone, Trideceth-9 PG-Amodimethicone and PEG/PPG-22/24 Dimethicone,
(25) Polyoxyalkylenated and/or (poly)glycerolated silanes, in particular chosen from Bis-PEG-18 Methyl Ether Dimethyl Silane and Bis-PEG-18 Methyl Ether Dimethyl Silane,
(26) Polyoxyethylenated or (poly)glycerolated acrylate copolymers, in particular the copolymer having the INCI name: Acrylate/Palmeth-25 Acrylate Copolymer,
(27) Polyoxyalkylenated alkanediols such as PEG-8 Caprylyl Glycol,
(28) Polyoxyethylenated rapeseed amides and sterols, in particular chosen from PEG-4 Rapeseed Amide and PEG-5 Rapeseed Sterol,
(29) Polyoxyethylenated lanolins such as Laneth-15,
(30) Polyoxyethylenated fatty acid esters of sorbitol such as PEG-40 Sorbitan Peroleate,
(31) Polyoxyethylenated glycerolated esters such as Glycereth-25 PCA Isostearate,
(32) Polyoxyethylenated or (poly)glycerolated acrylate copolymers, in particular the copolymer having the INCI name: Acrylate/Palmeth-25 Acrylate Copolymer, and
(33) mixtures thereof.

According to a particular embodiment, the compound(s) Y according to the invention is preferably present in a content equal or greater than 0.5% by weight, preferably equal or greater than 0.8% by weight, preferably equal or greater than 1% by weight, more particularly equal or greater than 2% by weight relative to the total weight of the composition containing it (them).

According to a particular embodiment, the compound(s) Y according to the invention is preferably present in a content from about 0.5 to about 30% by weight, preferably from about 0.75 to about 25% by weight, preferably from about 1 to about 25% by weight, and preferably from about 2 to about 15% by weight, relative to the total weight of the composition containing it (them), including all ranges and subranges therebetween such as, for example, 3 to 8% by weight, 0.5% to 7.5% by weight, 2 to 5% by weight, etc.

According to a preferential embodiment of the invention, the mole ratio of the reactive hydroxyl groups (OH) of the polyphenol(s) X to the hydroxyl groups of the compound(s) Y that are reactive with those of the polyphenol(s) X preferably ranges from 1/3 to 20 (20/1), preferably from 1/2 to 15 (15/1), and preferably from 3/4 to 4 (4/1)

### Solvent System

According to the present invention, compositions comprising a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application are provided.

Suitable C2-C5 monoalcohols include ethanol, propanol, butanol, pentanol, isopropanol, isobutanol and isopentanol. Ethanol is particularly preferred.

Preferably, the C2-C5 monoalcohol(s) is/are present in the compositions of the present invention in an amount effective to inhibit formation of a precipitate of compound X and compound Y in the composition prior to application. Preferably, the C2-C5 monoalcohol(s) is/are present in the compositions of the present invention in amounts ranging from about 5% to about 75%, preferably from about 10% to about 65%, preferably from about 20% to about 55%, and preferably from about 25% to about 50%, by weight, based on the total weight of the composition, including all ranges and subranges in between such as, for example, about 25% to about 65%.

According to preferred embodiments, the compositions of the present invention further comprise water. Preferably, the compositions comprise at least about 10% water by weight, preferably in amounts from about 10% to about 75%, preferably from about 15% to about 65%, preferably from about 20% to about 55%, and preferably from about 25% to about 50%, by weight, based on the total weight of the composition, including all ranges and subranges therebetween such as, for example, about 35% to about 55%.

Preferably, the solvent component of the compositions of the present invention consists essentially of, or consists of, water and C2-C5 monoalcohols. Preferably, the solvent component of the composition is “free of,” “devoid of” or “substantially free of” solvents other than water and C2-C5 monoalcohols.

According to preferred embodiments, the compositions of the present invention are devoid of, free of, or substantially free of, oils.

According to preferred embodiments, the compositions of the present invention are devoid of, free of, or substantially free of, waxes.

### Chelating Agent

According to preferred embodiments of the present invention, compositions further comprising at least one chelating agent are provided. Such chelating agents are known in the art and described, for example, in “Chelating agents” Kirk Othmer Encyclopedia of Chemical Technology, Vol. 5 pp. 708-739, published in 2003 and subsequent editions.

Suitable chelating agents include, but are not limited to, polyphosphates, aminocarboxylic acids, 1,3-diketones, hydroxycarboxylic acids, polyamines, amino alcohols, heterocyclic aromatic bases, aminophenols, Schiff's bases, tetrapyrroles, sulfur compounds, synthetic macrocyclic compounds, polymers and phosphonic acids. Preferably, chelating agents are selected from aminocarboxylic acids.

Specific examples of suitable chelating agents include, but are not limited to, ethylenediaminetetraacetic acid (EDTA), tetrasodium etidronate, tetrasodium pyrophosphate, pentasodium ethylenediamine tetramethylene phosphonate, sodium staminate, tetrasodium glutamate diacetate, tetraacetic acid, DPTA (1,3-diaminopropanetetraacetic acid), and mixtures thereof.

According to preferred embodiments, the compositions of the invention comprise chelating agent(s) in an amount ranging from about 0.0001 to about 5%, preferably from about 0.0002 to about 3%, preferably from about 0.002 to about 1%, and preferably from about 0.005 to 1%, by weight, based on the total weight of the composition, including all ranges and subranges therebetween such as, for example, 0.002 to 0.007%, 0.001 to 4%, 0.00025 to 1.25%, etc.

### Ascorbic Acid Compound

According to the present invention, compositions further comprising at least one ascorbic acid compound are provided. The ascorbic acid compound can be vitamin c (ascorbic acid) or substituted versions of vitamin c. Ascorbic acid compound(s) can function as antioxidants in the invention compositions and, thus, are preferably added in antioxidant effective amounts.

Suitable ascorbic acid compounds include esters of ascorbic acid (Vitamin C) which include, but are not limited to, water-soluble or fat-soluble ester(s) of ascorbic acid, such as for example tetrahexyldecyl ascorbate, ascorbyl palmitate, magnesium ascorbyl phosphate, ascorbyl glucoside, glucosamine ascorbate, ascorbyl acetate, and mixtures thereof.

According to preferred embodiments, the compositions of the invention comprise ascorbic acid compound(s) in an amount ranging from about 0.01 to about 5%, preferably from about 0.05 to about 3%, preferably from about 0.1 to about 1%, and preferably from about 0.25 to 0.75%, by weight, based on the total weight of the composition, including all ranges and subranges therebetween such as, for example, 0.1 to 3%, 0.2 to 1%, 0.075 to 0.75%, etc.

Preferably, the antioxidant component of the compositions of the present invention consists essentially of, or consists of, ascorbic acid compound(s).

Preferably, the antioxidant component of the composition is “free of,” “devoid of” or “substantially free of” antioxidants other than ascorbic acid compound(s), in particular sulfites and/or bisulfite antioxidants.

### Gelling Agent

According to preferred embodiments of the present invention, compositions optionally further comprising at least one gelling agent are provided. Suitable gelling agents include any gelling agent capable of providing thickening to a high-alcohol content solvent system such as for example acrylic acid (co) polymers, such as high molecular weight homo- or co-polymers comprising acrylic acid, optionally crosslinked with a polyalkenyl polyether, including some polymers identified as “carbomer” as well as amphiphilic polymers. Preferably, the gelling agent is an amphiphilic polymer.

The gelling agents, preferably amphiphilic polymers, may comprise at least one ethylenically unsaturated monomer, preferably containing a sulphonic group, in freeform or partially or totally neutralized form.

The amphiphilic polymers may comprise at least one hydrophobic portion. The hydrophobic portion present in these polymers preferably contains from 6 to 50 carbon atoms, preferably from 6 to 22 carbon atoms, preferably from 6 to 18 carbon atoms and preferably from 12 to 18 carbon atoms, including all ranges and subranges therebetween.

The amphiphilic polymers may have a molar mass ranging from 50,000 g/mole to 10,000,000 g/mole, preferably from 80,000 g/mole to 8,000,000 g/mole, and preferably from 100,000 g/mole to 7,000,000 g/mole.

The amphiphilic polymers may be based on at least one ethylenically unsaturated hydrophilic monomer A and on at least one hydrophobic monomer B. Preferably, the monomer A comprises a strong acid function, in particular a sulphonic acid or phosphonic acid function. The hydrophobic monomer B comprises at least one hydrophobic radical, chosen from: saturated or unsaturated C₆-C₁₈ linear alkyl radicals (for example, n-hexyl, n-octyl, n-decyl, n-hexadecyl, n-dodecyl or oleyl); branched alkyl radicals (for example, isostearic) or cyclic alkyl radicals (for example, cyclododecane or adamantane); C₆-C₁₈ fluoro or alkylfluoro radicals (for example, the group of formula —(CH₂)₂—(CF₂)₉—CF₃); a cholesteryl radical or radicals derived from cholesterol (for example, cholesteryl hexanoate); aromatic polycyclic groups, for instance naphthalene or pyrene; and silicone or alkylsilicone or alkylfluorosilicone radicals. Among these radicals, linear and branched alkyl radicals are preferred.

The amphiphilic polymers may be water-soluble or water-dispersible in neutralized form.

The amphiphilic polymers may be crosslinked. The crosslinking agents may be chosen from, for example, the polyolefinically unsaturated compounds commonly used for crosslinking polymers obtained by free-radical polymerization. According to one preferred embodiment of the invention, the crosslinking agent is chosen from methylenebisacrylamide, allyl methacrylate or trimethylolpropane triacrylate (TMPTA). The degree of crosslinking preferably ranges from 0.01 mol % to 10 mol %, and preferably from 0.2 mol % to 2 mol %, relative to the polymer, including all ranges and subranges therebetween.

The amphiphilic polymers may be homopolymers or copolymers.

The amphiphilic polymers can be partially or totally neutralized with a mineral base (for example, sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures thereof.

The amphiphilic polymers may be water-soluble or water-dispersible homopolymers such as, for example, optionally cross-linked polymers of sodium 2-acrylamido-2-methylpropane sulfonate acid such as that used in the commercial product SIMULGEL 800 (CTFA name: Sodium Polyacryloyldimethyl Taurate), cross-linked polymers of ammonium 2-acrylamido-2-methyl propane sulfonate acid (INCI name: AMMONIUM POLYACRYLDIMEHYLTAURAMIDE) such as the product sold under the tradename HOSTACERIN AMPS@ by Clariant.

The amphiphilic polymers may be chosen from crosslinked or non-crosslinked amphiphilic polymers of 2acrylamido-2-methylpropanesulphonic (AMPS) acid and of at least one ethylenically unsaturated monomer comprising at least one hydrophobic portion containing from 6 to 30 carbon atoms, preferably from 6 to 22 carbon atoms, preferably from 6 to 18 carbon atoms and preferably from 12 to 18 carbon atoms, including all ranges and subranges therebetween.

Suitable examples of amphiphilic polymers include, but are not limited to, hydrophobically-modified sulfonic acid copolymers such as Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Aristoflex SNC (crosslinked ethoxylated AMPS/C16-C18), Aristoflex LNC (noncrosslinked AMPS/C12-C14), and mixtures thereof.

Preferably, if present, the gelling agent(s) is/are present in the compositions of the present invention in amounts ranging from about 0.05 to about 5% by weight, preferably from 0.1 to 2.5% by weight, preferably from 0.3 to 2% and preferably from 0.5 to 1% by weight, all weights based on the weight of the composition as a whole, including all ranges and subranges therebetween such as, for example, 0.1 to 1.5%, 0.25 to 1.25%, 0.4 to 0.75%, etc.

### Oil Phase

According to embodiments of the present invention, the compositions of the present invention may optionally further comprise at least one oil. “Oil” means any non-aqueous medium which is liquid at ambient temperature (25° C.) and atmospheric pressure (760 mm Hg). Suitable oils can be volatile or non-volatile.

Suitable oils include volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Suitable oils include non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Suitable oils include synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; pentaerythritol esters; and synthetic ethers containing from 10 to 40 carbon atoms.

If present, the oil(s) is/are present in the compositions of the present invention in an amount ranging from about 0.1% to about 20% by weight, more preferably from about 0.4% to about 15% by weight, and preferably from about 0.5% to about 10% by weight, based on the total weight of the composition, including all ranges and subranges within these ranges.

According to preferred embodiments, however, compositions of the present invention are substantially free of, devoid of, or free of oils.

According to preferred embodiments, however, compositions of the present invention are substantially free of, devoid of, or free of volatile oils such as, for example, isododecane.

According to preferred embodiments, compositions of the present invention are substantially free of, devoid of, or free of silicone oils such as, for example, dimethicone.

### Coloring Agents

According to preferred embodiments of the present invention, compositions optionally further comprising at least one coloring agent are provided.

According to this embodiment, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, ß-carotene, soybean oil, Sudan Brown, DC Yellow 11, DC Violet 2, DC Orange 5, annatto, and quinoline yellow.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agents may be present in compositions of the present invention in an amount insufficient to provide visual color to the compositions (that is, the color of compositions of the present invention are a result of factor(s) other than the presence of coloring effective amounts of coloring agent, such as for example hydrolysis of polyphenol X which can provide color to the compositions) and/or in an amount insufficient to alter the visual color characteristics of a color cosmetic composition (for example, a makeup composition such as a foundation) when the composition of the present invention is used in conjunction with the color cosmetic composition as a primer or setter composition. Preferably, compositions of the present invention are free, substantially free, or devoid of coloring agents as defined above.

### Additional Additives

The composition of the invention can also comprise any additive usually used in the field under consideration. For example, film forming agents, waxes, dispersants such as poly(12-hydroxystearic acid), sunscreens, preserving agents, fragrances, fillers, neutralizing agents, cosmetic and dermatological active agents, moisturizers, silicone elastomers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002). However, it is to be understood that preferred embodiments of the present invention include compositions which are “free,” substantially free” or “devoid” of the ingredients discussed in this paragraph such as film forming agents and waxes.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium and should be able to be applied to the eyelashes of human beings.

According to preferred embodiments of the present invention, methods of caring for, and/or making up, keratinous material by applying compositions of the present invention to the keratinous material in an amount sufficient to care for, and/or to make up, the keratinous material are provided. Similarly, preferred embodiments of the present invention include methods of priming keratinous material for a cosmetic composition and/or of setting a cosmetic composition which has been previously applied to keratinous material by applying compositions of the present invention over keratinous material prior to application of a cosmetic composition (priming) and/or by applying compositions of the present invention over a cosmetic composition previously-applied to keratinous material (setting).

Preferably, “making up” the keratin material includes applying at least one coloring agent to the keratin material (in either the composition itself or in a color coat composition applied either over or under the composition as described above) in an amount sufficient to provide color to the keratin material.

In accordance with the preceding preferred embodiments, the compositions of the present invention are applied topically to the desired keratinous material. The compositions may be applied to the desired area as needed, preferably once or twice daily, more preferably once daily and then preferably allowed to dry before subjecting to contact such as with clothing or other objects (for example, a color coat composition or a topcoat applied over the composition). Preferably, the composition is allowed to dry for about 1 minute or less, more preferably for about 45 seconds or less.

According to preferred embodiments of the present invention, methods of stabilizing polyphenol X with respect to reducing aggregation as defined above are provided, wherein the composition, in particular a setter composition for cosmetics, comprises (1) at least one polyphenol X comprising at least two different phenol groups, (2) at least one (poly)glycerolated and/or polyoxyalkylenated nonionic compound Y, and (3) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application, wherein the method comprises adding at least one chelating agent and at least one ascorbic acid compound to the composition during preparation of the composition in an amount sufficient to stabilize the polyphenol X with respect to reducing aggregation as defined above in the composition are provided. The amount of the at least one chelating agent and at least one ascorbic acid compound added to the composition during preparation of the composition to stabilize the polyphenol are preferably those amounts discussed above in connection these ingredients.

According to preferred embodiments of the present invention, kits comprising, as separate compositions in one or more containers within the kits, (A) a composition, in particular a setter composition for cosmetics, comprising (1) at least one polyphenol X comprising at least two different phenol groups, (2) at least one (poly)glycerolated and/or polyoxyalkylenated nonionic compound Y, (3) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application, (4) at least one chelating agent, and (5) at least one ascorbic acid compound; and (B) at least one other composition such as a color coat composition comprising at least one colorant are provided. Preferably, the composition further comprises at least one gelling agent. Also preferably, the composition is a gel composition and/or has a pH less than 7.

Compositions (A) and (B) may be contained in different portions or sections of the same container within the kit. However, compositions (A) and (B) may also be in different containers with the kit.

According to preferred embodiments of the present invention, sets, as applied onto keratinous material, comprising (A) at least one first layer of at least one composition, in particular a setter composition for cosmetics, comprising (1) at least one polyphenol X comprising at least two different phenol groups, (2) at least one (poly)glycerolated and/or polyoxyalkylenated nonionic compound, (3) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application, (4) at least one chelating agent, and (5) at least one ascorbic acid compound; and (B) at least one second layer of a color coat composition comprising at least one colorant, where the at least one second layer is applied over (where the composition of the invention is a primer composition) or under (where the composition of the invention is a setter composition) the at least one first layer, are provided. Preferably, the composition further comprises at least one gelling agent. Also preferably, the composition is a gel composition and/or has a pH less than 7.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example I—Composition Preparation

Compositions were formulated by mixing the ingredients in following Table.

| | Inventive | Inventive | Inventive | Comparative | Comparative |
|---|---|---|---|---|---|
| | 1 | 2 | 3 | 1 | 2 |
| WATER | 48.05 | 47.55 | 48.05 | 48.30 | 48.05 |
| ETHANOL | 39 | 39 | 39 | 39 | 39 |
| TANNIC ACID | 5 | 5 | 5 | 5 | 5 |
| PEG SURFACTANT | 5 | 5 | 5 | 5 | 5 |
| AMPS THICKENER | 0.7 | 0.7 | 0.7 | 0.7 | 0.7 |
| SILICA SILYLATE | 2 | 2 | 2 | 2 | 2 |
| ASCORBYL GLUCOSIDE | 0.25 | 0.75 | 0 | 0 | 0 |
| ASCORBIC ACID | 0 | 0 | 0.25 | 0 | 0 |
| NAICINAMIDE | 0 | 0 | 0 | 0 | 0.25 |
| CHELATOR | 0.002 | 0.005 | 0.002 | 0 | 0.002 |
| TOTAL | 100 | 100 | 100 | 100 | 100 |

### Example II—Testing Protocols

### A. Aggregation Test

A composition is placed into a 45° C. oven for 4 weeks or 8 weeks. The composition is visually observed for aggregation in the composition. Visual aggregation within the composition indicates composition instability.

### B. Color Evaluation Testing

A composition is placed into a 45° C. oven for 4 weeks or 8 weeks. Color can be evaluated upon removal from the oven.

First, the color of the composition can be visually observed. Compositions containing different antioxidants and/or chelating agents can be compared to determine color differences, with lighter colors indicating better composition stability, in particular better polyphenol X stability within the composition. In this way, the effect on composition stability, in particular polyphenol X stability, of different antioxidants and/or chelating agents can be determined.

Second, colorimetry analysis can be performed to more quantitatively determine composition color and thus composition stability, in particular polyphenol X stability within the composition. For example, the composition can be deposited on white cards, coating the white card with a thickness of 6 mils. After a set amount of time to allow the deposit (or coating) to dry (for example, 1-4 hours), a color spectrometer can be used to determine the color value of areas of the white card with or without the deposit (or coating). The color for both areas can be recorded using L, a, b values as is known in the art. Color difference values ΔE can be calculated using differences in L, a, and b values in the white and coated/deposited areas of the card as follows:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

### Example III—Testing

Compositions were prepared in accordance with the Table above. These compositions were placed into a 45° C. oven for period of 4 weeks and 8 weeks. ΔE values were determined as described above.

Invention Composition 1: ΔE=2.57 (4 weeks); ΔE=3.78 (8 weeks). Invention Composition 2: ΔE=2.96 (4 weeks); ΔE=3.51 (8 weeks). Invention Composition 3: ΔE=7.52 (4 weeks); for 8 weeks the sample become liquid, and was difficult coat the card with the thickness needed (6 mil) to have accurate readings.

Comparative Composition 1: ΔE=5.55 (4 weeks); ΔE=6.25 (8 weeks). Comparative Composition 2: ΔE=3.60 (4 weeks); ΔE=6.34 (8 weeks).

These results indicate that invention compositions containing both chelating agent and ascorbic acid compounds were substantially more stable than compositions lacking both ingredients.

Invention Composition 1 and 2 contained ascorbyl glucoside and chelator, and both had limited color changes and had no visible aggregations.

Invention Composition 3 contained ascorbic acid and chelator. It was a darker composition (red) indicating that Vitamin C does not provide as much color stability to compositions as esters of ascorbic acid, which was a surprising additional benefit of the esters of ascorbic acid. However, it did prevent aggregation of polyphenol compound X as described above.

Comparative composition 1 did not contain any chelating agent or ascorbic acid compound. It contained visible aggregations at both 4 weeks and 8 weeks. Also, an increase of aggregates in size and number in 8 weeks versus 4 week was observed. With brown aggregates increased, the film deposit (coating) lost consistency for accurate and lower color readings.

Comparative composition 2 contained niacinamide instead of ascorbic acid compound. It did contain chelating agent. It only slightly improved inhibition of color darkening properties, and aggregations were formed.

In contrast, a composition of the invention containing ascorbyl glucoside and chelating agent was noticeably lighter in color and did not contain visual aggregations, indicating better stability of polyphenol X. These results indicate that invention compositions containing both chelating agent and ascorbic acid compound were substantially more color stable than compositions containing only chelating agent.

1. A method of making up keratinous material comprising applying a color coat composition and a composition comprising (a) at least one polyphenol X comprising at least two different phenol groups; (b) at least one (poly)glycerolated and/or polyoxyalkylenated nonionic compound Y; (c) a solvent system comprising (i) water in an amount of at least about 10% by weight with respect to the total weight of the composition; and (ii) at least one C2-C5 monoalcohol in an amount effective to inhibit formation of a precipitate of compound X and compound Y in the composition prior to application; (d) at least one chelating agent; and (e) at least one ascorbic acid compound to the keratinous material.

2. The method of claim 1, wherein the composition is in the form of a gel composition.

3. The method of claim 2, wherein the composition further comprises at least one gelling agent which is an amphiphilic polymer.

4. The method of claim 1, wherein the at least one (poly)glycerolated and/or polyoxyalkylenated nonionic compound Y has a molar mass greater than 200 g/mol.

5. The method of claim 1, wherein the at least one polyphenol compound X is tannic acid.

6. The method of claim 1, wherein the at least one C2-C5 monoalcohol is ethanol.

7. The method of claim 1, wherein the at least one ascorbic acid compound is ascorbyl glucoside.

8. The method of claim 1, wherein the at least one ascorbic acid compound is ascorbic acid.

9. The method of claim 1, wherein the composition has a pH which is less than 7.

10. The method of claim 1, wherein the at least one polyphenol compound X is tannic acid, the at least one C2-C5 monoalcohol is ethanol, the at least one ascorbic acid compound is ascorbyl glucoside, wherein the composition has a pH which is less than 7 and wherein the composition is in the form of a gel composition.

11. The method of claim 1, wherein the composition is substantially free of coloring agents.

12. The method of claim 1, wherein the composition comprises (a) from about 0.5 to about 15% by weight of at least one polyphenol X comprising at least two different phenol groups; (b) from about 0.5 to about 15% by weight of at least one (poly)glycerolated and/or polyoxyalkylenated nonionic compound Y; (c) from about 25 to about 65% by weight of at least one C2-C5 monoalcohol; (d) from about 0.002 to about 1% by weight of at least one chelating agent; (e) from about 0.1 to about 3% by weight of at least one ascorbic acid compound; and (f) from about 20 to about 55% by weight of water, all weights being based on the total weight of the composition.

13. The method of claim 12, wherein the composition has a pH which is less than 7 and the composition is in the form of a gel.

14. The method of claim 1, comprising applying the color coat composition to the keratinous material to form an applied color coat composition, and applying the composition over the applied color coat composition.

15. The method of claim 1, comprising applying the composition to the keratinous material to form an applied composition, and applying the color coat composition over the applied composition.

16. The method of claim 1, wherein the keratinous material is skin or lips.
